(19) United States
(12) Patent Application Publication
(10) Pub. No.: US 2009/0312627 A1
(43) Pub. Date: Dec. 17, 2009

(54) Radio-labeled ingestible capsule

(76) Inventors: Laura A. Matott, East Aurora, NY (US); John R. Semler, Williamsville, NY (US); Mark Scott, London (GB)

Correspondence Address: PHILLIPS LYTLE LLP;INTELLECTUAL PROPERTY GROUP, 3400 HSBC CENTER, BUFFALO, NY, 14203-3509, US

(21) Appl. No.: 12/456,093

(22) Filed: Jun. 11, 2009

Related U.S. Application Data
(60) Provisional application No. 61/132,157, filed on Jun. 16, 2008.

(51) Int. Cl. A61B 6/12

(52) U.S. Cl. 600/424

(57) ABSTRACT

A method of determining the location of an ingested capsule comprising the steps of providing an ingestible capsule (15) having a shell (16) and a flexible fluid retaining sleeve (17) affixed to the sleeve and defining a chamber (19) between the shell and the sleeve, the fluid retaining sleeve having a fill port (30), providing a radioactive material (22) contained in a filling device (31) having an output port (32) adapted to engage the fill port of the sleeve, engaging the output port of the filling device with the fill port of the sleeve, moving the radioactive material through the output port and into the chamber of the sleeve through the sleeve fill port, sealing the fill port to provide a radio-labeled ingestible capsule, having a subject ingest the radio-labeled capsule, and screening the subject by gamma imagining to determine location of the radio-labeled capsule within the subject.

### CROSS-REFERENCE TO RELATED APPLICATIONS

This application claims the benefit of U.S. Provisional Patent Application No. 61/132,157, filed Jun. 16, 2008. The entire content of such application is incorporated by reference herein.

### TECHNICAL FIELD

The present invention relates to ingestible capsules and, more particularly, to a trackable radio-labeled ingestible capsule.

### BACKGROUND ART

Ingestible capsules are well known in the prior art and various capsules have been developed. These are generally small pill-like devices that can be ingested or swallowed by a patient. It is known that such capsules may include one or more sensors for determining physiological parameters of the gastrointestinal tract, such as sensors for detecting temperature, pH and pressure.

A number of methods of determining location of an ingestible capsule are known in the prior art. For example, it is known that signal strength or signal triangulation may be used to attempt to determine the location of an ingested capsule. However, the use of an RF signal has a number of disadvantages, including that it generally requires multiple antennas, various tissues may impact the signal differently, and patient movement may skew the results. It is also known that accelerometers may be used to attempt to determine location, but such methods also have disadvantages, such as drift, non-linear progression and rotational inaccuracy.

It is also known that certain physiological parameters may be associated with regions of the gastrointestinal tract. For example, a 1988 article entitled “Measurement of Gastrointestinal pH Profiles in Normal Ambulant Human Subjects” discloses pH measurements recorded by a capsule passing through the gastrointestinal tract. While pH has been correlated with transitions from the stomach to the small bowel (gastric emptying) and from the distal small bowel to the colon (ileo-caecal transition), often there are not significant pH variations correlated with certain regions of the gastrointestinal tract, and patients with gastrointestinal maladies may have abnormal readings.

It is known that colorectal transit time can be measured in the clinical setting using scintigraphy and radio-opaque markers. Colonic scintigraphy involves administering a radioactive substance to a subject and following the progression of the isotope with a gamma camera, at specific time points, through the gastrointestinal tract. This is considered the gold-standard technique, as it provides information regarding regional as well as colonic transit. Colonic transit time may also be measured uses radio-opaque markers. This technique is easier and inexpensive, although its reliability for determining regional colonic transit time is reduced.

### DISCLOSURE OF THE INVENTION

With parenthetical reference to the corresponding parts, portions or surfaces of the disclosed embodiment, merely for the purposes of illustration and not by way of limitation, the present invention provides a method of determining the location of an ingested capsule comprising the steps of providing an ingestible capsule (15) having a shell (16) and a flexible fluid retaining sleeve (17) affixed to the sleeve and defining a chamber (19) between the shell and the sleeve, the fluid retaining sleeve having a fill port (30), providing a radioactive material (22) contained in a filling device (31) having an output port (32) adapted to engage the fill port of the sleeve, engaging the output port of the filling device with the fill port of the sleeve, moving the radioactive material through the output port and into the chamber of the sleeve through the sleeve fill port, sealing the fill port to provide a radio-labeled ingestible capsule, having a subject ingest the radio-labeled capsule, and screening the subject by gamma imaging to determine location of the radio-labeled capsule within the subject.

The filling device may comprise a syringe and the output port may comprise a nozzle (32) on the syringe. The step of engaging the output port of the filling device with the fill port of the sleeve may comprise inserting the nozzle into the fill port of the sleeve. The step of moving the radioactive material through the output port and into the chamber of the sleeve through the sleeve fill port may comprise applying a force to a plunger (33) adapted to move axially relative to a barrel (34) of a syringe (31). The method may further comprise the step of trimming the sealed fill port and cleaning the capsule. The seal may be a heat seal and the method may further comprise the step of inspecting the heat seal under a microscope and inspecting the sleeve for air bubbles. The fill port may comprise a tubular tail, and the step of sealing the fill port to provide a radio-labeled ingestible capsule may comprise the steps of providing a heat sealer (35), clamping the tubular tail at a base location (36), inserting at least a portion of the tubular tail extending beyond the base location into the heat sealer, heat sealing such portion of the tubular tail, and removing the clamp. The radioactive material may be a liquid radio isotope. The step of screening the subject by gamma imaging to determine location of the capsule within the subject may comprise the steps of having a subject ingest a background isotope to illuminate the intestinal tract, gamma imaging the subject, and determining location of the capsule as a function of contrast between the radio-labeled capsule and the background isotope.

The capsule may comprise a pressure sensor (18) operatively arranged to sense pressure within the chamber. The method may further comprise the step of sensing pressure with the pressure sensor when the capsule is ingested by the subject. The capsule may comprise a pH sensor (23) operatively arranged to sense pH within the gastrointestinal tract of the subject. The method may further comprise the step of sensing pH with the pH sensor when the capsule is ingested by the subject. The step of gamma imaging the subject may be a function of pH or pressure measurements obtained from the pH or pressure sensor. The location of the capsule may be determined as a function of the gamma imaging, pressure measurements taken by the pressure sensor and pH measurements taken by the pH sensor. The method may further comprise the steps of recording pH measurements from a pH sensor on the capsule as a function of time as the capsule moves through at least a portion of the gastrointestinal tract of the subject, recording pressure measurements from a pressure sensor on the capsule as a function of time as the capsule moves through at least a portion of the gastrointestinal tract of the subject, deriving a pressure pattern as a function of time and the pressure measurements, deriving a pH pattern as a function of time and the pH measurements, and applying the gamma imaging as a function of the pH and pressure patterns. The method may further comprise the steps of recording pH measurements from a pH sensor on the capsule as a function of time as the capsule moves through at least a portion of the gastrointestinal tract of the subject, recording pressure measurements from a pressure sensor on the capsule as a function of time as the capsule moves through at least a portion of the gastrointestinal tract of the subject, deriving a pressure pattern as a function of time and the pressure measurements, deriving a pH pattern as a function of time and the pH measurements, and determining the location of the capsule as a function of the gamma imaging and the pH and pressure patterns.

In another aspect, the invention provides a radio-labeled ingestible capsule (15) comprising a shell, a flexible fluid retaining sleeve affixed to the shell and defining a chamber between the shell and the sleeve, a fluid retaining sleeve having a sealed fill port (40), and a liquid radio isotope (22) contained within the chamber, whereby the capsule may be located by gamma imaging. The capsule may further comprise a pressure sensor (18) operatively arranged to sense pressure within the chamber, whereby a contraction force on an outside surface of the sleeve produces a corresponding pressure change within the chamber. The shell may be rigid and the sleeve may be more elastic than the shell. The chamber may be filled with the liquid radio isotope to a base pressure and the sensor may sense an increase in pressure in the chamber above the base pressure. The shell may comprise a fluid port (25) communicating with the chamber and the sensor, and the chamber and fluid port may be operatively arranged such that the sensor senses an increase in pressure in the port. The sensor may comprise a piezoelectric bridge or an oscillator coil and diaphragm. The capsule may further comprise a plurality of ribs (20a-b) extending from the shell and supporting the sleeve. The ribs may be configured to form a plurality of subchambers (21a-c), whereby force on the sleeve produces a corresponding pressure in a subchamber that is channeled by the ribs towards the sensor. The sleeve may be stretched over the ribs. The capsule may further comprise a pH sensor (23).

Accordingly, the general object is to provide a device and method for determining the location of a capsule in a mammalian tract.

Another object is to provide a capsule that can be tracked using gamma imagining.

Another object is to provide a capsule that senses pressure or pH and can be tracked using gamma imagining.

Another object is to provide a capsule which can be radioactively marked just prior to ingestion.

These and other objects and advantages will become apparent from the foregoing and ongoing written specification, the drawings and the appended claims.

### BRIEF DESCRIPTION OF THE DRAWINGS

FIG. 1 is a perspective view of an embodiment of the improved capsule.

FIG. 2 is a longitudinal horizontal sectional view of the capsule shown in FIG. 1 being filled.

FIG. 3 is a view of the capsule shown in FIG. 2 being sealed.

FIG. 4 is a view of the capsule shown in FIG. 3 after sealing.

FIG. 5 is a view of the capsule shown in FIG. 4 after trimming of the sealed port.

FIG. 6 is a transverse horizontal sectional view of the capsule shown in FIG. 1, taken generally on line 6-6 of FIG. 1.

### DESCRIPTION OF THE PREFERRED EMBODIMENTS

At the outset, it should be clearly understood that like reference numerals are intended to identify the same structural elements, portions or surfaces, consistently throughout the several drawing figures, as such elements, portions or surfaces may be further described or explained by the entire written specification, of which this detailed description is an integral part. Unless otherwise indicated, the drawings are intended to be read (e.g., cross-hatching, arrangement of parts, proportion, degree, etc.) together with the specification, and are to be considered a portion of the entire written description of this invention. As used in the following description, the terms “horizontal”, “vertical”, “left”, “right”, “up” and “down”, as well as adjectival and adverbial derivatives thereof (e.g., “horizontally”, “rightwardly”, “upwardly”, etc.), simply refer to the orientation of the illustrated structure as the particular drawing figure faces the reader. Similarly, the terms “inwardly” and “outwardly” generally refer to the orientation of a surface relative to its axis of elongation, or axis of rotation, as appropriate.

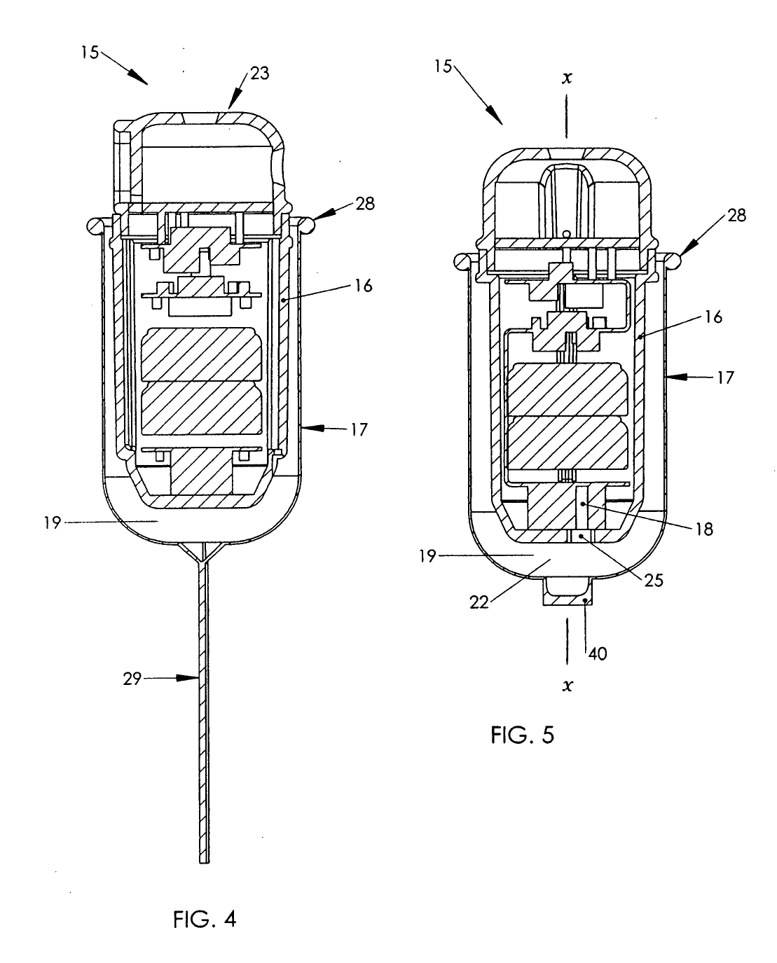

Referring now to the drawings, and more particularly to FIGS. 4-5 thereof, a radio-labeled capsule is generally indicated at 15. Capsule 15 is shown as being an elongated ellipsoid-shaped device, somewhat resembling a medicament capsule, and broadly includes a shell or casing 16, a flexible fluid retaining sleeve 17 affixed to the shell and defining a chamber 19 between the shell and the sleeve, a pressure sensor 18 operatively arranged to sense pressure within the chamber and communicating with the chamber through a fluid port 25, and a pH sensor 23 operatively arranged to sense pH.

The capsule generally has a hard shell or casing 16 which houses the transmitting electronics, battery compartment and sensors. Capsule 15 is adapted to be ingested, implanted, inserted or otherwise positioned within a mammalian body or tract to sense pressure within the body or tract and to transmit such pressure. As shown, plastic shell 16 is generally a cylindrical member elongated about axis x-x and having generally rounded closed ends. Shell 16 is generally provided to facilitate easy swallowing of the capsule and, in the preferred embodiment, is composed of a hard polyurethane plastic.

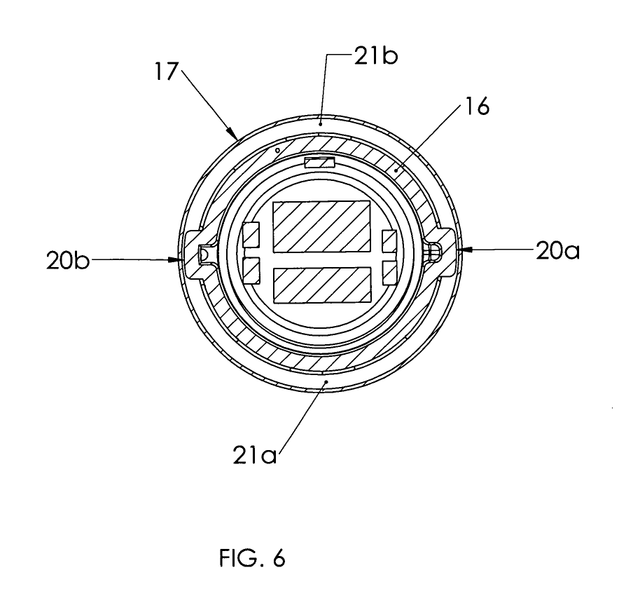

As shown in FIG. 6, shell 16 includes two opposed ribs 20a and 20b extending longitudinally along a portion of the outside cylindrical surface of the shell. Ribs 20a and 20b project radially beyond the outside cylindrical surface of the lower portion of shell 16.

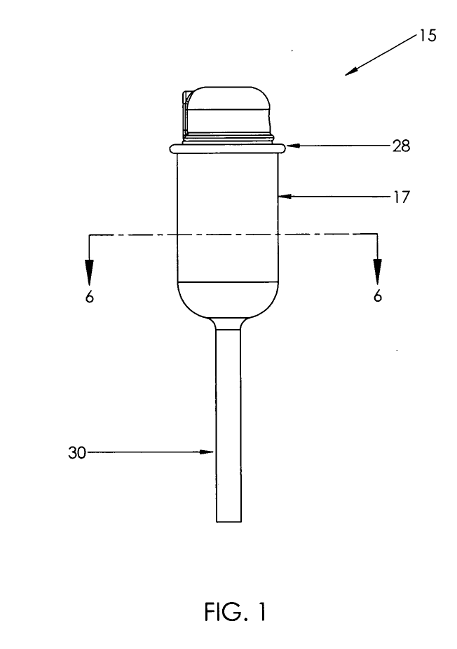

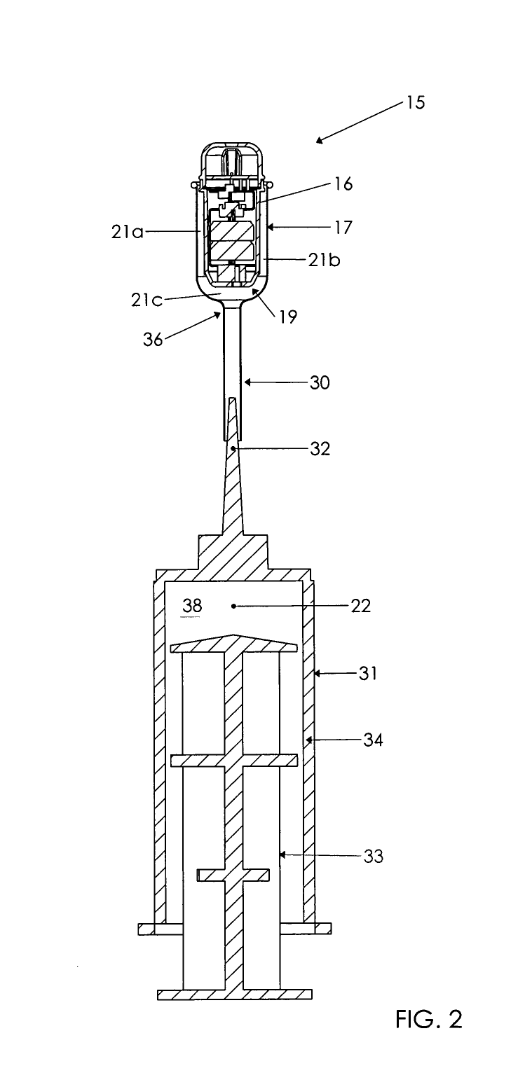

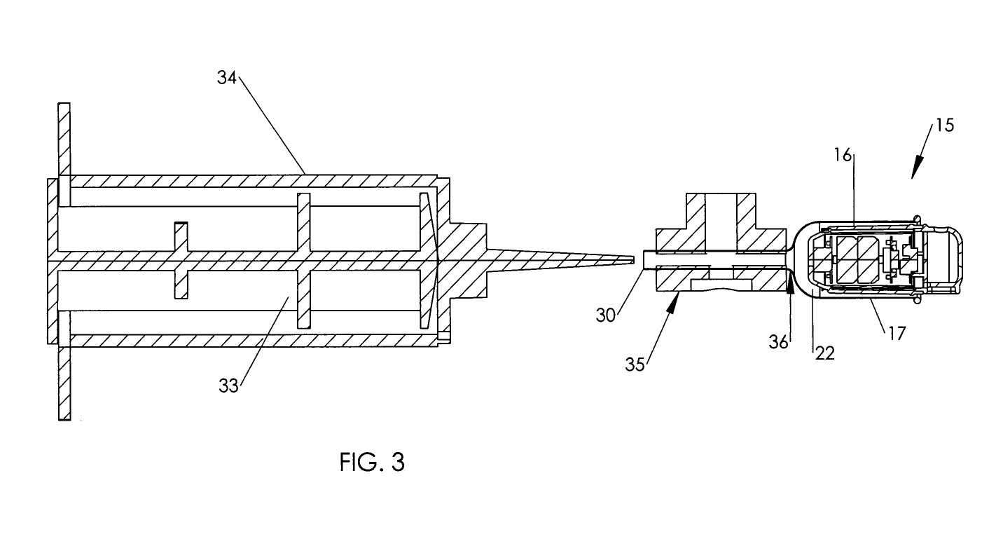

Sleeve 17 is stretched over ribs 20a and 20b of shell 16 and extends from attachment 28 down around the bottom two-thirds of shell 16. In the preferred embodiment, sleeve 17 does not extend over the entire shell 16 of capsule 15 and is composed of a polyurethane and polycarbonate blend, although sleeve 17 can be made of other elastomeric materials such as natural or synthetic rubber. As shown, sleeve 17 resembles a balloon, and the open end is rolled over to form an annular bead having a slightly smaller inner diameter than the rest of sleeve 17. Sleeve 17 extends around the outer annular surface of shell 16 at attachment point 28 by interference fit coupling and is secured in place by an adhesive. As shown in FIGS. 1-4, the end of sleeve 17 includes a filling port 30 through which chamber 19 between shell 16 and sleeve 17 may be filled with a radioactive nucleotide 22.

Sleeve 17 is configured and stretched over shell 16 so as to form a chamber 19. Sleeve 17 is adjusted on shell 16 such that the sleeve contacts and is held against ribs 20a and 20b. It is contemplated that this contact may be maintained by stretching the sleeve over the ribs and by the elasticity of sleeve 17, or alternatively the sleeve may be secured to the outer surface of ribs 20a and 20b by glue or other fastening means. Because of the configuration of shell 16, and in particular the use of ribs 20a and 20b, chamber 19 has three connected compartments or sub-chambers 21a, 21b and 21c. Sub-chambers 21a and 21b are defined by the longitudinal space between sleeve 17, shell 16 and ribs 20a and 20b. Sub-chamber 21c is defined by the space between the bottom end of sleeve 17 and the bottom end of shell 16. Sleeve 17 provides a semi-flexible containment area for radioactive fluid 22 and translates external force applied to the capsule to pressure sensor 18.

As shown in FIG. 4, shell 16 has an outer surface and an inner surface and sensor 18 is mounted and supported by the interior surface of shell 16. The bottom end of shell 16 includes a fluid port 25 which extends from chamber 19 into the interior of shell 16. Fluid port 25 allows radioactive fluid 22 in chamber 19 to communicate with pressure sensor 18.

Pressure sensor 18 is a conventional piezoelectric bridge. As fluid presses against the sensor's bridge, it creates an electric signal which corresponds to the pressure of fluid 22 in chamber 19. Pressure sensor 18 provides good linearity and allows for single point calibration. The GE Nova pressure sensor manufactured by GE Thermal Metrics, of 808 US Highway 1, Edison, N.J., may be used in the preferred embodiment. It is contemplated that other pressure sensors may be used. For example, the pressure sensor may comprise a diaphragm in communication with chamber 19, a non-ferrous disk, and an oscillatory coil and capacitor in parallel, which oscillate at a base frequency on the application of a current through the coil. The diaphragm is supported by the interior surface of shell 16, and an annular rim and a contact port is provided at the end of shell 16. The diaphragm extends across the interior end of the port. The diaphragm has a flexural modulus that is less then the flexural modulus of sleeve 17 and is capable of deflecting as a result of changing pressure in chamber 17. A non-ferrous disk is attached to the internal surface of the diaphragm. When the diaphragm deflects towards the coil, as a result of an increase in pressure in chamber 19, the non-ferrous disc moves towards the coil, which decreases the inductance and therefore increases the frequency of oscillation of the coil. This change in frequency corresponds to a given change in pressure in chamber 19.

Chamber 19 is filled with a radioactive nucleotide fluid 22. In the preferred embodiment, the fluid used is 10 MBq ⁵¹Cr-EDTA. Fluid 22 not only permits gamma tracking of capsule 15 but is also a non-compressible medium that forms part of the 360° degree force sensing mechanism for sensor 18.

On the opposite end of capsule 15 to pressure sensor 18 is pH sensor 23. In the preferred embodiment, pH sensor 23 is a conventional ISFET type pH sensor. ISFET stands for ion-selective field effect transistor and the sensor is derived from MOSFET technology (metal oxide screen field effect transistor). A current between a source and a drain is controlled by a gate voltage. The gate is composed of a special chemical layer which is sensitive to free hydrogen ions (pH). Versions of this layer have been developed using aluminum oxide, silicon nitride and titanium oxide. Free hydrogen ions influence the voltage between the gate and the source. The effect on the drain current is based solely on electrostatic effects, so the hydrogen ions do not need to migrate through the pH sensitive layer. This allows equilibrium, and thus pH measurement, to be achieved in a matter of seconds. The sensor is an entirely solid state sensor, unlike glass bulb sensors which require a bulb filled with buffer solution. Only the gate surface is exposed to the sample.

In the preferred embodiment, the capsule transmits sensed data at about 434 MHz and measures 26.8 mm long by 11.7 mm in diameter. A portable data receiver worn by the subject receives and stores data transmitted by the capsule. Software performs data analysis and presents a graphical data display of pH, pressure and temperature readings for analysis. After activation and ingestion, the capsule senses and transmits data for at least 120 hours after activation. The pH, pressure and temperature data are transmitted from within the GI tract to the data receiver. In the preferred embodiment, the range and accuracy of the sensors are generally 1 to 9 pH units with an accuracy of ±0.5 pH units, 0 to 350 mmHg with an accuracy of 5 mmHg, or 10% above 100 mmHg, and 25° to 49° C. with an accuracy of ±1° C. The data receiver contains rechargeable batteries and when seated in a docking station allows for battery charging and data download. Data is downloaded from the data receiver through the docking station via USB connection to a Windows PC compatible laptop.

Capsule 15 is labeled with a radioactive fluid just prior to ingestion. As shown, chamber 19 of capsule 15 is adapted to be filled immediately prior to ingestion with a liquid radio-isotope 22. A liquid radio isotope 22 is prepared and disposed within chamber 38 of syringe 31. Either syringe 31 is provided pre-filled, or syringe 31 is filled by the user by removing plunger 33 and attaching the end to an automated pump and reservoir of radioactive material. The radioactive fluid dispenser is turned on and the vacuum adjusted. A tip cap is applied to the end of the syringe so that material will not leak from nozzle 32. Syringe 31 is then filled with radioactive solution 22 until it is three quarters full. Plunger 33 is inserted into barrel 34 of syringe 31 and the tip cap of the syringe is removed and tapered nozzle 32 positioned for insertion into the end of port 30 in sleeve 17. Nozzle 32 is then inserted into the end of port 30 until nozzle 32 fills the cylindrical end of port 30. Any air in chamber 19 is evacuated by moving plunger 33 outwardly relative to barrel 34. When held properly, the evacuated air will rise through the radioactive material 22 in barrel 34 away from nozzle 32. Plunger 33 is then compressed inwardly relative to barrel 34 to apply a force on radioactive material 22 until chamber 19 of sleeve 17 is entirely filled with solution 22 to a fill pressure that will result in a desired pressure when the sleeve is sealed. In this embodiment, the seal pressure is between about 25 and 60 mmHg, and preferably about 40 mmHg. Forceps are then used to clamp port 30 at a base location 36 and nozzle 32 is removed from the end of port 30.

A conventional heat sealer 35 is then provided. The portion 29 of long tubular port 30 that extends beyond clamped location 36 is placed into the opposed jaws of sealer 35, and the heated jaws are then closed as close as possible to the base 36 of tubular port 30. The jaws are held together and a heat seal applied. The jaws are then released and the seal inspected for completeness. These steps may be repeated until a complete seal is applied to form a sealed tail 29, as shown in FIG. 4. As shown in FIG. 5, the end of sealed tail 29 is then trimmed to leave about one millimeter of length 40.

Capsule 15 is then washed and the surface of the capsule is wiped free of lint. The seal is inspected for completeness under a microscope. The user then gently squeezes the surface of sleeve 17, moving towards sealed port 40 to check for any liquid leaking out of seal 40. Chamber 19 is also inspected for air bubbles. There should be no air bubbles greater than one millimeter in diameter visible when sleeve 17 is held at a distance of six inches from the user.

The purpose of radio-labeling the capsule is to track the capsule in the gastrointestinal tract of a subject. In a first embodiment, the marked capsule is tracked by gamma imaging. In a second embodiment, a background isotope is used to help illuminate the intestinal tract, and the two markers contrast upon gamma imaging. The capsules path can thereby be plotted against the outline of the intestinal tract. In this embodiment, 4 MPq of ¹¹¹In-DTPA is administered orally and provides the background silhouette in which the capsule gamma localization can be determined as a function of time.

Capsule 15 is thereby a non-invasive, ingestible device that incorporates both pressure and pH sensors and can be tracked with conventional gamma imaging. After swallowing, the capsule progresses through the different regions of the gastrointestinal tract, simultaneously registering pH and pressure events. Changes in pH as well as pressure as a function of transit time can therefore be used to determine when to apply gamma imagining to the subject and/or to corroborate the location of the capsule as determined by gamma imaging.

As described in U.S. Patent Application Publication No. 2008/0064938 entitled “Method of Determining Location of an Ingested Capsule”, the entire contents of which are incorporated herein my reference, pH readings and pressure readings from ingested capsule 15 are plotted against time.

Based on reference data, a substantial variation or increase in pH indicates passage of the capsule from the stomach to the small intestine, often referred to as gastric emptying. A latter variation in pH suggests movement of the capsule from the ileum to the caecum. This significant pH drop is seen some hours after gastric emptying and is due to the capsule moving from the ileum to the caecum, a transition referred to as the ileo-caecal junction.

Average pressure readings from the capsule are plotted against transit time. The number of contractions over a baseline for a given time interval, five minutes in the preferred embodiment, plotted against the same overall time period are also plotted. In the preferred embodiment, a contraction is designated by an increase in pressure over 10 mmHg and the subsequent return below 10 mmHg. However, it is contemplated that gastrointestinal contractions may be determined based on other variations in pressure or baselines other than 10 mmHg.

A variation in the frequency of contractions can generally be found to occur at a time corresponding to the gastric emptying suggested by the graph of pH. This correlation between the variation in frequency of contractions and the variation in pH is used as a reference to confirm that the capsule has moved from the stomach to the small bowel. A further and more substantial variation in contractions occurs at a time corresponding to the ileo-caecal junction suggested by the graph of pH. This correlation between the variation in frequency of contractions and the variation in pH is used as a reference to determine that the capsule has moved from the ileum to the caecum of the subject.

Motility index as used herein is the area under the curve (or the integral of pressure over a time region) divided by the size of the time region. A variation in motility index may also be used in the preferred embodiment as a reference to confirm that the capsule has moved from the ileum to the caecum of the subject. Also, a variation in motility index may be used as a reference with pH variation to confirm that the capsule has moved from the stomach to the small intestine.

Readings from a subject may therefore be compared to a reference template to determine general location. Thus, a change in pH and a change in either frequency of contractions or motility index that correlates with the variations in the template may be used to determine location. By first determining the general location of capsule 15 based on pH and/or pressure patterns taken by the capsule, the user can select to apply gamma imaging to the subject only when the capsule is in that part of the gastrointestinal tract of interest to the user. Thus, gamma imaging does not have to be applied as often to track the capsule, but instead is applied more selectively as a function of where the pH and/or pressure measurements from the capsule indicate its location. This allows for selective gamma imaging.

In addition, the pH and pressure measurements may be used with gamma imaging to more accurately determine the movement of capsule 15 through the gastrointestinal tract. Selective gamma imaging may be used to corroborate the location of capsule 15 indicated by the pH or pressure patterns from the subject.

The present invention contemplates that many changes and modifications may be made. Therefore, while the presently-preferred form of the capsule and method have been shown and described, and a number of alternatives discussed, persons skilled in this art will readily appreciate that various additional changes and modifications may be made without departing from the spirit of the invention, as defined and differentiated by the following claims.

What is claimed is:

1. A method of determining the location of an ingested capsule comprising the steps of:
providing an ingestible capsule having a shell and a flexible fluid retaining sleeve affixed to said shell and defining a chamber between said shell and said sleeve, said fluid retaining sleeve having a fill port;
providing a radioactive material contained in a filling device having an output port adapted to engage said fill port;
engaging said output port of said filling device with said fill port of said sleeve;
moving said radioactive material through said output port and into said chamber of said sleeve through said sleeve fill port;
sealing said fill port to provide a radio-labeled ingestible capsule;
having a subject ingest said radio-labeled capsule; and
screening said subject by gamma imaging to determine location of said radio-labeled capsule within said subject.

2. The method set forth in claim 1, wherein said filling device comprises a syringe and said output port comprises a nozzle.

3. The method set forth in claim 2, wherein said step of engaging said output port of said filling device with said fill port of said sleeve comprises inserting said nozzle into said fill port of said sleeve.

4. The method set forth in claim 2, wherein said step of moving said radioactive material through said output port and into said chamber of said sleeve through said sleeve fill port comprises applying a force to a plunger adapted to move axially relative to a barrel of a syringe.

5. The method set forth in claim 1, and further comprising the steps of trimming said sealed fill port and cleaning said ingestible capsule.

6. The method set forth in claim 1, wherein said seal is a heat seal.

7. The method set forth in claim 6, and further comprising the step of inspecting said heat seal under a microscope and inspecting said sleeve for air bubbles.

8. The method set forth in claim 7, wherein said sleeve is inspected for air bubbles that are greater than about 1 mm in diameter.

9. The method set forth in claim 1, wherein said fill port comprises a tubular tail.

10. The method set forth in claim 9, wherein said step of sealing said fill port to provide a radio-labeled ingestible capsule comprises the steps of:
providing a heat sealer;
clamping said tubular tail at a base location;
inserting at least a portion of said tubular tail extending beyond said base location into said heat sealer;
heat sealing said portion of said tubular tail; and
removing said clamp.

11. The method set forth in claim 1, wherein said radioactive material is a liquid radioisotope.

12. The method set forth in claim 1, wherein said step of screening said subject by gamma imaging to determine location of said capsule within said subject comprises the steps of:
having said subject ingest a background isotope to illuminate the intestinal tract;
gamma imaging said subject; and
determining said location of said capsule as a function of contrast between said radio-labeled capsule and said background isotope.

13. The method set forth in claim 1, wherein said capsule comprises a pressure sensor operatively arranged to sense pressure within said chamber,

14. The method set forth in claim 13, and further comprising the step of sensing pressure with said pressure sensor when said capsule is ingested by said subject.

15. The method set forth in claim 1, and further comprising the steps of
recording pH measurements from a pH sensor on said capsule as a function of time as said capsule moves through at least a portion of the gastrointestinal tract of said subject;
recording pressure measurements from a pressure sensor on said capsule as a function of time as said capsule moves through at least a portion of said gastrointestinal tract of said subject;
deriving a pressure pattern as a function of time and said pressure measurements;
deriving a pH pattern as a function of time and said pH measurements;
applying gamma imaging as a function of said pH and pressure patterns.

16. The method set forth in claim 1, and further comprising the steps of
recording pH measurements from a pH sensor on said capsule as a function of time as said capsule moves through at least a portion of the gastrointestinal tract of said subject;
recording pressure measurements from a pressure sensor on said capsule as a function of time as said capsule moves through at least a portion of said gastrointestinal tract of said subject;
deriving a pressure pattern as a function of time and said pressure measurements;
deriving a pH pattern as a function of time and said pH measurements;
determining said location of said capsule as a function of said gamma imaging and said pH and pressure patterns.

17. A radio-labeled ingestible capsule comprising:
a shell;
a flexible fluid retaining sleeve affixed to said shell and defining a chamber between said shell and said sleeve;
said fluid retaining sleeve having a sealed fill port; and
a liquid radioisotope contained within said chamber;
whereby said capsule may be located by gamma imaging.

18. The capsule set forth in claim 17, and further comprising a pressure sensor operatively arranged to sense pressure within said chamber, whereby a contraction force on an outside surface of said sleeve produces a corresponding pressure change within said chamber.

19. The capsule set forth in claim 18, wherein said shell is rigid and said sleeve is more elastic than said shell.

20. The capsule set forth in claim 18, wherein said chamber is filled with said liquid radioisotope to a base pressure and said sensor senses an increase in pressure in said chamber above said base pressure.

21. The capsule set forth in claim 18, wherein said shell comprises a fluid port communicating with said chamber.

22. The capsule set forth in claim 21, wherein said sensor, said chamber and said port are operatively arranged such that said sensor senses an increase in pressure in said port.

23. The capsule set forth in claim 18, wherein said sensor is a piezoelectric bridge or an oscillator coil and diaphragm.

24. The capsule set forth in claim 18, and further comprising a plurality of ribs extending from said shell and supporting said sleeve.

25. The capsule set forth in claim 24, wherein said ribs are configured to form a plurality of sub-chambers, whereby a force on said sleeve produces a corresponding pressure in a sub-chamber that is channeled by said ribs towards said sensor.

26. The capsule set forth in claim 24, wherein said sleeve is stretched over said ribs.
